(19) United States
(12) Patent Application Publication
(10) Pub. No.: US 2019/0323066 A1
(43) Pub. Date: Oct. 24, 2019

(54) Rapid Methods for the Detection of Microbial Resistance

(71) Applicant: Longhorn Vaccines and Diagnostics, LLC, Bethesda, MD (US)

(72) Inventors: Luke T. Daum, San Antonio, TX (US); Gerald W. Fischer, Bethesda, MD (US)

(73) Assignee: Longhorn Vaccines and Diagnostics, LLC, Bethesda, MD (US)

(21) Appl. No.: 16/389,217

(22) Filed: Apr. 19, 2019

Related U.S. Application Data
(60) Provisional application No. 62/660,402, filed on Apr. 20, 2018.

(51) Int. Cl. C12Q 1/689

(52) U.S. Cl. CPC C12Q 1/689

(57) ABSTRACT

The invention is directed to methods, kits, compositions for the detection of microbial resistance in bacteria, viruses, parasites, fungus, and other microbes. The methods of the invention are both rapid and inexpensive thereby allowing for appropriate treatment of large numbers of individual patients.

### REFERENCE TO RELATED APPLICATIONS

This application claims priority to U.S. Provisional Application No. 62/660,402 filed Apr. 20, 2018, the entirety of which is incorporated by reference herein.

### BACKGROUND

### 1. Field of the Invention

The present invention is directed to methods, kits, and compositions for the detection and identification of resistance in microbes and, in particular, methods that that are both rapid, straightforward to operate, and inexpensive.

### 2. Description of the Background

Mycobacterium tuberculosis (MTB) is a pathogenic bacterial species in the family Mycobacteriaceae and the causative agent of most cases of tuberculosis (TB). Another species of this genus is M. leprae, the causative agent of leprosy. MTB was first discovered in 1882 by Robert Koch, M. tuberculosis has an unusual, complex, lipid rich, cell wall which makes the cells impervious to Gram staining. Acid-fast detection techniques are used to make the diagnosis instead. The physiology of M. tuberculosis is highly aerobic and requires significant levels of oxygen to remain viable. Primarily a pathogen of the mammalian respiratory system, MTB is generally inhaled and, in five to ten percent of individuals, will progress to an acute pulmonary infection. The remaining individuals will either clear the infection completely or the infection may become latent. It is not clear how the immune system controls MTB, but cell mediated immunity is believed to play a critical role (Svenson et al., Human Vaccines, 6-4:309-17, 2010). Common diagnostic methods for TB are the tuberculin skin test, acid-fast stain and chest radiographs.

Well over ninety percent of individuals infected with MTB remain outwardly healthy with no demonstrable symptoms. These individuals are classified as latently infected and are a reservoir from which active MTB cases continue to develop (“reactivation tuberculosis”). Latent infection is generally defined as the absence of clinical symptoms of TB in addition to a delayed hypersensitivity reaction to the purified protein derivative of MTB used in tuberculin skin test or a T-cell response to MTB-specific antigens. The absence of an understanding of latency and thereby reliable control measures for treatment, makes latent tuberculosis infections a serious problem.

M. tuberculosis requires oxygen to proliferate and does not retain typical bacteriological stains due to high lipid content of its cell wall. While mycobacteria do not fit the Gram-positive category from an empirical standpoint (i.e., they do not retain the crystal violet stain), they are classified as acid-fast Gram-positive bacteria due to their lack of an outer cell membrane.

M. tuberculosis has over one hundred strain variations and divides every 15-20 hours, which is extremely slow compared to other types of bacteria that have division times measured in minutes (Escherichia coli can divide roughly every 20 minutes). The microorganism is a small bacillus that can withstand weak disinfectants and survive in a dry state for weeks. The cell wall of MTB contains multiple components such as peptidoglycan, mycolic acid and the glycolipid lipoarabinomannan. The role of these moieties in pathogenesis and immunity remain controversial. (Svenson et al., Human Vaccines, 6-4:309-17, 2010).

MTB infection is spread by airborne droplet nuclei, which contain the pathogen expelled from the lungs and airways of those with active TB. The infectious droplet nuclei are inhaled and lodge in the alveoli and in the alveolar sac where M. tuberculosis is taken up by alveolar macrophages. These macrophages invade the subtending epithelial layer, which leads to a local inflammatory response initiating formation of the granuloma, the hallmark of tuberculosis disease. That results in recruitment of mononuclear cells from neighboring blood vessels, thus providing fresh host cells for the expanding bacterial population. However, these macrophages are unable to digest the bacteria because the cell wall of the bacteria prevents the fusion of the phagosome with a lysosome. Specifically, M. tuberculosis blocks the bridging molecule, early endosomal autoantigen 1 (EEA1); however, this blockade does not prevent fusion of vesicles filled with nutrients. As a consequence, bacteria multiply unchecked within the macrophage. The bacteria also carry the UreC gene, which prevents acidification of the phagosome, and also evade macrophage-killing by neutralizing reactive nitrogen intermediates.

With the arrival of lymphocytes, the granuloma acquires a more organized, stratified structure. Development of an immune response takes about 4-6 weeks after the primary infection is indicated by a positive DTH (delayed type hypersensitivity) reaction to Tuberculin. The balance between host immunity (protective and pathologic) and bacillary multiplication determines the outcome of infection. An encounter with MTB is classically regarded to give rise to three possible outcomes. The first possible outcome, which occurs in a minority of the population, is the rapid development of active TB and associated clinical symptoms. The second possible outcome, which occurs in the majority of infected individuals, do not include disease symptoms. These individuals develop an effective acquired immune response and are considered to have a “latent infection.” A portion of latently infected individuals over time will reactivate and develop active TB. Roughly ten percent of these infected individuals (mainly infants or children) will develop progressive clinical disease referred to as primary active TB. Primary TB usually occurs within 1-2 years after the initial infection. This results from local bacillary multiplication and spread in the lung and/or blood. Spread through the blood can seed bacilli in various tissues and organs. Post-primary, or secondary, TB can occur many years after infection owing to loss of immune control and the reactivation of bacilli. The immune response of the patient results in a pathological lesion that is characterized by localized, often extensive tissue damage, and cavitations. The characteristic features of active post-primary TB can include extensive lung destruction with cavitation, positive sputum smear (most often), and upper lobe involvement, however these are not exclusive. Patients with cavitary lesions (i.e., granulomas that break through to an airway) are the main transmitters of infection. In latent TB, the host immune response is capable of controlling the infection but falls short of eradicating the pathogen. Latent TB is defined on solely on the evidence of sensitization by mycobacterial proteins that is a positive result in either the Tuberculin skin test (TST) reaction to purified protein derivative of MTB or an in vitro interferon-gamma (IFN-γ) release assay to MTB-specific antigens, in the absence of clinical symptoms or isolated bacteria from the patient.

The BCG vaccine (Bacille de Calmette et Guérin) against tuberculosis is prepared from a strain of the attenuated, but live bovine tuberculosis bacillus, Mycobacterium bovis. This strain lost its virulence to humans through in vitro subculturing in Middlebrook 7H9 media. As the bacteria adjust to subculturing conditions, including the chosen media, the organism adapts and in doing so, loses its natural growth characteristics for human blood. Consequently, the bacteria can no longer induce disease when introduced into a human host. However, the attenuated and virulent bacteria retain sufficient similarity to provide immunity against infection of human tuberculosis. The effectiveness of the BCG vaccine has been highly varied, with an efficacy of from zero to eighty percent in preventing tuberculosis for duration of fifteen years, although protection seems to vary greatly according to geography and the lab in which the vaccine strain was grown. This variation, which appears to depend on geography, generates a great deal of controversy over use of the BCG vaccine yet has been observed in many different clinical trials. For example, trials conducted in the United Kingdom have consistently shown a protective effect of sixty to eighty percent, but those conducted in other areas have shown no or almost no protective effect. For whatever reason, these trials all show that efficacy decreases in those clinical trials conducted close to the equator. In addition, although widely used because of its protective effects against disseminated TB and TB meningitis in children, the BCG vaccine is largely ineffective against adult pulmonary TB, the single most contagious form of TB.

A 1994 systematic review found that the BCG reduces the risk of getting TB by about fifty percent. There are differences in effectiveness, depending on region due to factors such as genetic differences in the populations, changes in environment, exposure to other bacterial infections, and conditions in the lab where the vaccine is grown, including genetic differences between the strains being cultured and the choice of growth medium.

The duration of protection of BCG is not clearly known or understood. In studies showing a protective effect, the data are inconsistent. The MRC study showed protection waned to 59% after 15 years and to zero after 20 years; however, a study looking at Native Americans immunized in the 1930s found evidence of protection even 60 years after immunization, with only a slight waning in efficacy. Rigorous analysis of the results demonstrates that BCG has poor protection against adult pulmonary disease, but does provide good protection against disseminated disease and TB meningitis in children. Therefore, there is a need for new vaccines and vaccine antigens that can provide solid and long-term immunity to MTB.

The role of antibodies in the development of immunity to MTB is controversial. Current data suggests that T cells, specifically CD4⁺ and CD8⁺ T cells, are critical for maximizing macrophage activity against MTB and promoting optimal control of infection (Slight et al, JCI 123(2):712, February 2013). However, these same authors demonstrated that B cell deficient mice are not more susceptible to MTB infection than B cell intact mice suggesting that humoral immunity is not critical. Phagocytosis of MTB can occur via surface opsonins, such as C3, or nonopsonized MTB surface mannose moieties. Fc gamma receptors, important for IgG facilitated phagocytosis, do not seem to play an important role in MTB immunity (Crevel et al., Clin Micro Rev. 15(2), April, 2002; Armstrong et al., J Exp Med. 1975 Jul. 1; 142(1):1-16). IgA has been considered for prevention and treatment of TB, since it is a mucosal antibody. A human IgA monoclonal antibody to the MTB heat shock protein HSPX (HSPX) given intra-nasally provided protection in a mouse model (Balu et al, J of Immun. 186:3113, 2011). Mice treated with IgA had less prominent MTB pneumonic infiltrates than untreated mice. While antibody prevention and therapy may be hopeful, the effective MTB antigen targets and the effective antibody class and subclasses have not been established (Acosta et al, Intech, 2013).

Cell wall components of MTB have been delineated and analyzed for many years. Lipoarabinomannan (LAM) has been shown to be a virulence factor and a monoclonal antibody to LAM has enhanced protection to MTB in mice (Teitelbaum, et al., Proc. Natl. Acad. Sci. 95:15688-15693, 1998, Svenson et al., Human Vaccines, 6-4:309-17, 2010). The mechanism whereby the MAB enhanced protection was not determined and the MAB did not decrease bacillary burden. It was postulated that the MAB possibly blocked the effects of LAM induced cytokines. The role of mycolic acid for vaccines and immune therapy is unknown. It has been used for diagnostic purposes, but has not been shown to have utility for vaccine or other immune therapy approaches. While MTB infected individuals may develop antibodies to mycolic acid, there is no evidence that antibodies in general, or specifically mycolic acid antibodies, play a role in immunity to MTB.

Antibiotic resistance is becoming more and more of a problem for treating MTB infections. Beginning with the first antibiotic treatment for TB in 1943, some strains of the TB bacteria developed resistance to the standard drugs through genetic changes. The BCG vaccine against TB does not provide protection from acquiring TB to a significant degree. In fact, resistance accelerates if incorrect or inadequate treatments are used, leading to the development and spread of multidrug-resistant TB (MDR-TB). Incorrect or inadequate treatment may be due to use of the wrong medications, use of only one medication (standard treatment is at least two drugs), not taking medication consistently or for the full treatment period (treatment is required for several months). Treatment of MDR-TB requires second-line drugs (e.g., fluoroquinolones, aminoglycosides, and others), which in general are less effective, more toxic and much more expensive than first-line drugs. If these second-line drugs are prescribed or taken incorrectly, further resistance can develop leading to extreme-drug resistant TB (XDR-TB). Resistant strains of TB are already present in the population, so MDR-TB and XDR-TB are directly transmitted from an infected person to an uninfected person. Thus, a previously untreated person can develop a new case of MDR-TB or XDR-TB absent prior infection and/or treatments. This is known as primary MDR-TB or XR-TB and is responsible for up to 75% of new TB cases. Acquired MDR-TB and XR-TB develops when a person with a non-resistant strain of TB is treated inadequately, resulting in the development of antibiotic resistance in the TB bacteria infecting them. These people can in turn infect other people with MDR-TB.

Drug-resistant TB caused an estimated 480,000 new TB cases and 250,000 deaths in 2015, and accounts for about 3.3% of all new TB cases worldwide. These resistant forms of TB bacteria, either MDR-TB or rifampin-resistant TB, cause 3.9% of new TB cases and 21% of previously treated TB cases. Globally, most drug-resistant TB cases occur in South America, Southern Africa, India, China, and areas of the former Soviet Union.

Treatment of MDR-TB requires treatment with second-line drugs, usually four or more anti-TB drugs for a minimum of 6 months, and possibly extending for 18-24 months if rifampin resistance has been identified in the specific strain of TB with which the patient has been infected. Under ideal program conditions, MDR-TB cure rates can approach 70%. XR-TB infection requires even more-robust and prolonged treatment regiments.

Thus there is a strong need to rapidly characterize drug resistant strains of MTB such that personalized drug therapies can be implemented. In addition, as MDR-TB, and also TB, is a major problem in third world countries, reducing expense is critical.

### SUMMARY OF THE INVENTION

The present invention overcomes the problems and disadvantages associated with current strategies and designs and provide new tools and methods for detecting microbial resistance.

One embodiment of the invention is directed to methods for determining microbial resistance comprising: extracting nucleic acid from a biological sample containing a microbe; optionally, performing a quantitative PCR to increase quantity and/or assess a microbial nucleic acid; providing a collection of primer pairs, wherein each primer pair contains a common sequence and a variable sequence, wherein the variable sequence hybridizes to multiple regions of the microbial genome responsible for the expression of resistance genes; combining the microbial nucleic acid with the collection of primers pairs in a PCR to generate a series of amplicons; linking the series of amplicons to a unique sequence that is specific for the biological sample; providing a second collection of primer pairs, wherein each primer pair contains a sequence that is complimentary to the unique sequence and a sequence that hybridizes to one or more of the amplicon; combining the linked series of amplicons with the second collection of primers pairs in a PCR to generate a second series of amplicons; sequencing the second series of amplicons; comparing the sequences of the second series of amplicons with the sequence of a wild-type sequence of the microbial nucleic acid; and identifying one or more mutations of the resistance genes of the biological sample. Preferably, the microbial resistance comprises resistance to an antibiotic. Preferable, the biological sample is a bodily fluid, a nasal discharge, a sputum sample, blood, a tissue sample, a biopsy, a culture sample, or a combination thereof. Preferably, the microbe is a bacterium, a virus, a parasite, and/or a fungus. Preferably, the microbe is Mycobacterium tuberculosis. Preferably, the biological sample is collected in a transport medium containing guanidine, a reducing agent, a chelator, a nonionic detergent, and a buffer. Preferably, the biological sample is sterilized. Preferably, extracting is performed by chemical or mechanical treatment of the biological sample. Preferably, the primer pairs of the collection and/or the second collection are each from about 20 to about 35 nucleotides in length. Preferably, the common and/or the unique sequence is from about 8 to 15 nucleotides in length. Preferably, each of the multiple regions of the microbial genome are each from about 2 kb to about 20 kb in length. Preferably, the microbial genome contains four or more different antibiotic resistance genes such as, for example, rpoB, katG, gyrA, and pncA of Mycobacterium tuberculosis. Preferably, linking if performed by ligating the common sequence to the 5′ terminus of each amplicon of the series of amplicons. Preferably, linking if performed by ligating the unique sequence to the 5′ terminus of each amplicon of the second series of amplicons. Preferably, the polymerase chain reaction is an RT-PCR. Preferably, the amplicons of the series of amplicons generated and/or second series of amplicons generated are from 50 to 1,000 nucleotides in length, more preferably, from 100 to 500 nucleotides in length. Preferably, the amplicons of the series of amplicons generated and/or second series of amplicons generated are diluted in a buffer and a portion of the diluted amplicons subjected to sequencing. Preferably, sequencing is performed by ion torrent or next generation sequencing and also preferably sequencing of amplicons of the second series of amplicons is performed in one step. Preferably, the method is performed in about 24 to about 36 hours, more preferably in less than about 24 hours.

Another embodiment of the invention is directed to treating a patient infected with Mycobacteria comprising: performing the method of the invention disclosed herein, wherein the biological sample is obtained from the patient; and treating the patient with one or more drugs or drug combinations. Preferably, the time period from performing the method to treating the patient is less than 48 hours, more preferably the time period from performing the method to treating the patient is less than 36 hours, and more preferably the time period from performing the method to treating the patient is less than 24 hours.

Another embodiment of the invention is directed to kits for the detection of microbial resistance comprising: a transport media for collection of a biological sample; a collection of primer pairs for a PCR reaction, wherein each primer pair contain a common sequence and a variable sequence, wherein the variable sequence hybridizes to multiple regions of the microbial genome responsible for the expression of resistance genes; a second collection of primer pairs, wherein each primer pair of the second collection contains a sequence that is complimentary to the unique sequence; and a PCR mixture comprising: a heat-stable polymerase, deoxynucleotide tri phosphates comprising about equal amounts of dATP, dCTP, dGTP and/or dTTP; a chelating agent; a salt; a buffer; and a stabilizing agent.

Another embodiment of the invention is directed to methods for determining microbial resistance in multiple biological samples comprising: extracting nucleic acid from each of the multiple biological samples, each containing the same microbe; optionally, separately performing a quantitative PCR to increase quantity and/or assess the microbial nucleic acid of one or more of the multiple biological samples; providing a collection of primer pairs, wherein each primer pair contain a common sequence and a variable sequence, wherein the variable sequence hybridizes to multiple regions of the microbial genome responsible for the expression of resistance genes; separately combining each microbial nucleic acid extracted with the collection of primers pairs in a PCR to generate a series of amplicons for each biological sample; separately linking each series of amplicons to a unique sequence that is specific for the biological sample; providing second collections of primer pairs, one second collection for each series of amplicons generated, wherein each second collection contains a sequence that is complimentary to the unique sequence and a sequence that hybridizes to one or more of the amplicons; separately combining the linked series of amplicons with the second collections in a PCR to generate a second series of amplicons for each biological sample; pooling all the second series of amplicons for each biological sample generated; sequencing the pooled amplicons; comparing the sequences of the pooled amplicons with the sequence of a wild-type sequence of the microbial nucleic acid; and identifying one or more mutations in the resistance genes for each of the biological samples.

Other embodiments and advantages of the invention are set forth in part in the description, which follows, and in part, may be obvious from this description, or may be learned from the practice of the invention.

### DESCRIPTION OF THE INVENTION

Mycobacterium tuberculosis (MTB) is the causative agent of tuberculosis (TB) and approximately one third of the world population is infected with Mycobacterium tuberculosis (MTB). Increasing cases of multidrug-resistant (MDR), and extensively drug-resistant (XDR) MTB strains continue to circulate, particularly throughout Asia, Africa and Eastern Europe. While MDR strains are those resistant to antibiotics rifampin (RIF) and isoniazid (INH), XDR strains exhibit additional resistance to fluoroquinolone's (FQ) and at least one injectable aminoglycoside drug, e.g., amikacin, kanamycin or capreomycin. TB in one form or another, inflicts inflects approximately a third of our planet with drug resistant strains becoming more common.

Targeted sequencing is a process for analyzing a specific sequence of nucleotides in an organism that offers several advantages over the traditional Next-generation sequencing (NGS) approach. Specifically, it enables a focused and more sensitive approach for generating reliable high quality (i.e., Q>30) data and adequate coverage depth. Next-generation sequencing using targeted amplification includes of a series of discrete steps that uniquely contribute to the overall quality of a data set. Standardized sequencing metrics provide information about the accuracy of sample processing, including library preparation, base calling, read alignment, and variant calling. Base calling accuracy is measured by the Phred quality score (Q score) and is typically utilized to assess the accuracy base-calls by the sequencer. Cost and time are also important to the NGS workflow. The need for a quality method the ensure reproducibility and reduces time-to-result, without the use of ancillary and expensive equipment is of critical importance, particularly in resource-limited environments.

It has been surprisingly discovered that microbial resistance can be determined from a biological sample quickly and inexpensively, which are critical concerns for treatment. Determination is uncomplicated involving only basic laboratory techniques with nominal training. In addition, there are no harsh or dangerous chemical, devices, or other materials. Thus, risks to health care personnel are as minimal as possible.

One embodiment of the invention is directed to a method for determining microbial resistance of a microbe such as, for example, a bacterial, viral, parasitic or fungal infection. Preferably, the microbe is Mycobacterium tuberculosis (MTB) or an Influenza virus.

Preferably, the biological sample is collected in a transport medium and more preferably an aqueous transport medium containing guanidine, a reducing agent, a chelator, a nonionic detergent, and a buffer. Preferred aqueous transport medium is PRIMESTORE™ (Longhorn Vaccines and Diagnostics, LLC, Bethesda, Md.). Preferably, the biological sample is sterilized. Preferably, extracting is performed by chemical or mechanical treatment of the biological sample.

Resistance is determined from an analysis of a biological sample obtained from a patient suspected of being infected. The method comprises: extracting nucleic acid from a biological sample. Preferably, the biological sample, for example, is a bodily fluid, a nasal discharge, a sputum sample, blood, a tissue sample, a biopsy, or a combination thereof. Alternatively, the biological sample can be obtained from a culture to which the biological sample was applied for growth and/or development such as, for example, an agar plate or other growth support, a broth, a suspension, and/or in vitro cell culture.

Optionally, the extracted nucleic acids may be treated by a quantitative PCR to increase the quantity and/or assess a microbial nucleic acid. The nucleic acids from the biological sample or from the quantitative PCR are combined with a collection of primer pairs, wherein each primer pair contains a common sequence and a variable sequence, wherein the variable sequence hybridizes to multiple regions of the microbial genome known or expected to be responsible for the expression of the microbe's resistance genes. The common sequence is identical for all of the primers and preferable links to the 5′terminus of each primer. Preferably the primer pairs of the collection are each from about 15 to about 35 nucleotides in length, more preferably about 18 to 25 nucleotides. Primers may be larger or smaller as well. Preferably the common sequence is from about one third to one half of the length of the primer. preferably from about 8 to 15 nucleotides in length.

A PCR is performed on the microbial nucleic acid and first series of primer pairs to generate a series of amplicons. The polymerase chain reaction may be PCR for DNA sequences, or reverse transcribed PCR (RT-PCR) for RNA sequences. As there is a common sequence on each primer, the series of amplicons generated will each retain that common sequence and that common sequences identifies the amplicons as generated from a specific biological sample.

The amplicons generated are each linked to a unique sequence that is again specific for the biological sample. Preferably the unique sequence is from about 4 to 20 nucleotides in length, but may be longer or shorter as desired. More preferable the unique sequence is from 6-10 nucleotides in length. Preferably, linking if performed by ligating the common sequence to the 5′ terminus of each amplicon of the series of amplicons. Preferably, linking if performed by ligating the unique sequence to the 5′ terminus of each amplicon of the second series of amplicons.

A second collection of primer pairs are provided that are preferably from about 15 to about 35 nucleotides in length, more preferably about 18 to 25 nucleotides (although smaller or larger primers may be utilized). Each primer pair contains a sequence that is complimentary to the unique sequence and a sequence that hybridizes to one or more of the amplicon. The linked series of amplicons with the second collection of primers pairs are combined in a PCR to generate a second series of amplicons. This second series of amplicons are sequenced and sequences determined compared with the sequence of a wild-type sequence of the microbial nucleic acid. One or more mutations of the resistance genes of the biological sample can be identified from the comparison with the wild-type sequence. Preferably, the microbial resistance comprises resistance to an antibiotic or other drug or drug mixture.

Another embodiment of this invention is performing a first PCR separately on multiple biological samples and performing all of the steps of the method as outlined above. Prior to sequencing, the various amplicons generated can be combined, optionally diluted, and sequenced together. The mutations identified can be easily identified to a specific biological sample, and thus patient, because each unique sequence identifies with the specific biological sample.

Preferably, the amplicons of the series of amplicons generated and/or second series of amplicons generated are from 50 to 1,000 nucleotides in length, more preferably, from 100 to 500 nucleotides in length. Preferably, the amplicons of the series of amplicons generated and/or second series of amplicons generated are diluted in a buffer and a portion of the diluted amplicons subjected to sequencing. Preferably, sequencing is performed by ion torrent or next generation sequencing and also preferably sequencing of amplicons of the second series of amplicons is performed in one step. Preferably, the method is performed in about 24 to about 36 hours, more preferably in less than about 24 hours.

For MTB, preferably, the microbial genome contains four or more different antibiotic resistance genes such as, for example, rpoB, katG, gyrA, and pncA. For Influenza, drug resistance regions are the HA and NA regions of the Influenza genome.

Another embodiment of the invention is directed to treating a patient infected with Mycobacteria comprising: performing the method of the invention disclosed herein, wherein the biological sample is obtained from the patient; and treating the patient with one or more drugs or drug combinations. Preferably, the time period from performing the method to treating the patient is less than 48 hours, more preferably the time period from performing the method to treating the patient is less than 36 hours, and more preferably the time period from performing the method to treating the patient is less than 24 hours.

Another embodiment of the invention is directed to a kit for the detection of microbial resistance comprising: a transport media for collection of a biological sample; a collection of primer pairs for a PCR reaction, wherein each primer pair contain a common sequence and a variable sequence, wherein the variable sequence hybridizes to multiple regions of the microbial genome responsible for the expression of resistance genes; a second collection of primer pairs, wherein each primer pair of the second collection contains a sequence that is complimentary to the unique sequence; and a PCR mixture comprising: a heat-stable polymerase, deoxynucleotide tri phosphates comprising about equal amounts of dATP, dCTP, dGTP and/or dTTP; a chelating agent; a salt; a buffer; and a stabilizing agent.

Another embodiment of the invention is directed to a method for determining microbial resistance in multiple biological samples comprising: extracting nucleic acid from each of the multiple biological samples, each containing the same microbe; optionally, separately performing a quantitative PCR to increase quantity and/or assess the microbial nucleic acid of one or more of the multiple biological samples; providing a collection of primer pairs, wherein each primer pair contain a common sequence and a variable sequence, wherein the variable sequence hybridizes to multiple regions of the microbial genome responsible for the expression of resistance genes; separately combining each microbial nucleic acid extracted with the collection of primers pairs in a PCR to generate a series of amplicons for each biological sample; separately linking each series of amplicons to a unique sequence that is specific for the biological sample; providing second collections of primer pairs, one second collection for each series of amplicons generated, wherein each second collection contains a sequence that is complimentary to the unique sequence and a sequence that hybridizes to one or more of the amplicons; separately combining the linked series of amplicons with the second collections in a PCR to generate a second series of amplicons for each biological sample; pooling all the second series of amplicons for each biological sample generated; sequencing the pooled amplicons; comparing the sequences of the pooled amplicons with the sequence of a wild-type sequence of the microbial nucleic acid; and identifying one or more mutations in the resistance genes for each of the biological samples.

Although the invention is generally described in reference to human infection by Mycobacterium tuberculosis, as is clear to those skilled in the art the methodology and compositions are generally and specifically applicable to the treatment and prevention of many other diseases and infections in many other subjects (e.g., cattle, horses, sheep, cats, dogs, farm animals, pets, etc.) and most especially diseases wherein the causative agent is of viral, bacterial, fungal and parasitic origins. Microbes where the methods of the invention can be applied to determine and identify resistance include, for example, Streptococcus spp., Pseudomonas spp., Shigella spp., Yersinia spp. (e.g., Y. pestis), Clostridium spp. (e.g., C. botulinum, C. difficile), Listeria spp., Staphylococcus spp., Salmonella spp., Vibrio spp., Chlamydia spp., Gonorrhea spp., Syphilis spp., MRSA, Streptococcus spp., Escherichia spp. (e.g., E.coli), Pseudomonas spp., Aeromonas spp., Citrobacter spp (e.g., C. freundii, C. braaki), Proteus spp., Serratia spp., Klebsiella spp., Enterobacter spp., Chlamydophila spp., Mycobacterium spp., MRSA (Methicillin-resistant Staphylococcus aureus), and Mycoplasma spp. (e.g., Ureaplasma parvum, Ureaplasma urealyticum). Virus on which the methods of the invention can be applied include Rubella virus, Hepatitis virus, Herpes Simplex virus, retrovirus, varicella zoster virus, human papilloma virus, parvovirus, HIV. Parasitic infections on which the method of the invention can be applied include, for example, Plasmodium spp., Leishmania spp., Guardia spp., endoparasites, protozoan, and helminth spp. Fungal infections to which the methods of the invention can be applied include, for example, Cryptococci, aspergillus and candida. Diseases caused by microbes to which the methodology can be applied include sepsis, colds, flu, gastrointestinal infections, sexually transmitted diseases, immunodeficiency syndrome, nosocomial infections, Celiac disease, inflammatory bowel disease, inflammation, multiple sclerosis, auto-immune disorders, chronic fatigue syndrome, Rheumatoid arthritis, myasthenia gravis, Systemic lupus erythematosus, and infectious psoriasis.

The following examples illustrate embodiments of the invention, but should not be viewed as limiting the scope of the invention.

### EXAMPLES

### Example 1

Targeted sequencing is a process for analyzing a specific sequence of nucleotides in an organism that offers several advantages over the traditional Next-generation sequencing (NGS) approach. Specifically, it enables a focused and more sensitive approach for generating reliable high quality (i.e., Q>30) data and adequate coverage depth. Next-generation sequencing using targeted amplification includes of a series of discrete steps that uniquely contribute to the overall quality of a data set. Standardized sequencing metrics provide information about the accuracy of sample processing, including library preparation, base calling, read alignment, and variant calling. Base calling accuracy is measured by the Phred quality score (Q score) and is typically utilized to assess the accuracy base-calls by the sequencer. Cost and time are also important to the NGS workflow. The need for a quality method the ensure reproducibility and reduces time-to-result, without the use of ancillary and expensive equipment is of critical importance, particularly in resource-limited environments.

Mycobacterium tuberculosis (MTB) is the causative agent of tuberculosis (TB). Increasing cases of multidrug-resistant (MDR), and extensively drug-resistant (XDR) MTB strains continue to circulate, particularly throughout Asia, Africa and Eastern Europe. While MDR strains are those resistant to antibiotics rifampin (RIF) and isoniazid (INH), XDR strains exhibit additional resistance to fluoroquinolone's (FQ) and at least one injectable aminoglycoside drug, e.g., amikacin, kanamycin or capreomycin. TB in one form or another, inflicts inflects approximately a third of our planet with drug resistant strains becoming more common.

There is an urgent need to rapidly characterize drug resistant strains such that personalized drug therapies can be implemented. Standard whole-genome genetic analysis using next-generation sequencing (NGS) has enabled rapid genome analysis with minimal sample preparation time (2-4 days) at moderate costs when multiple samples are analyzed per run. The Illumina MiSeq platform can sequence up to 24 whole MTB genomes per run with an average reproducible coverage depth of ˜30 times. However, the standard NGS workflow requires the utilization of expensive equipment and reagents for library preparation which are cost prohibitive in many areas of the world. For example, the Nextera XT Library Prep Kit (FC-131-1024) uses Illumina's proprietary ‘tagmentation-fragmentation’ (referred as ‘tag and frag’) technology for precise size selection and library normalization prior to instrument loading on Illumina platforms. The kit requires frozen enzyme storage and an expanded in-depth workflow. Most importantly, the kit has a very high list price for 24 reactions.

The methods and kit as disclosed and described herein circumvents the use of this expensive method by utilizing a highly sensitive and specific ‘targeted’ amplification approach the employs hybrid oligonucleotide primers that perform three important steps: 1) Targeted amplification of the region of interest. In this example, amplification of key TB genes (rpoB, katG, gyrA, pncA) in specific regions where drug resistance-conferring mutations are known to arise. 2) through PCR the targeted oligonucleotides generate amplicons of the desired length (i.e., 100-500 nucleotides) which circumvents the need to size-selection using a conventional system or cumbersome excision using gel electrophoresis. 3) the five prime ends of the target oligonucleotides contain the requisite oligonucleotide ‘adaptor’ sequences that enable a subsequent downstream amplification for ‘barcode’ or ‘indexing’ multiple patient samples on a single run. This reduces costs associated with NGS and enables a unified analysis of genomic data for rapid screening of drug resistance-conferring mutations in MTB genes.

Thus, the developed methodology reduces the workflow, the need for ancillary equipment, and the costs for Illumina or other NGS library preparation kits and reagents. A brief overview of the workflow using drug resistance MTB gene targeting is below:

1. Collection: Samples, e.g., MGIT, LJ culture or primary sputum are collected, preferably in PrimeStore MTM.

2. Extraction: Samples are extracted preferably using PrimeXtact Kit according to manufacturer's recommendations (conventional extraction systems that can be used include, for example, Qiagen, Roche MagNApure).

3. Confirmatory qPCR: A quantitative PCR determines the quality and quantity of the collected/extracted sample and provides a metric for determining downstream NGS success.

4. Targeted NGS amplification: Primer pairs produce amplicons between 400 and 500 nucleotides in length. The primers contain Longhorn's optimized target specific sequences for generation of regions of the rpoB, katG, gyrA, and pncA genes. The regions target areas where the most prevalent resistance-conferring mutations are known to occur. More importantly, the primers contain overhand adapter sequences that are appended to the primer pair sequences for compatibility with Illumina index and sequencing adapters.

5. Prepare Library: Illumina Sequencing barcodes (indexes) can be added using the incorporated adaptor sequence from step 4 above. This entails a secondary PCR (12 cycles only; approximately 30 minutes). A PCR clean-up is performed to remove PCR reagents and purify the libraries.

6. Quantification and Normalization: Libraries are quantified using a small bench-top Qubit fluorometer and subsequently diluted to ˜5 nM. Importantly, up to 96 libraries can be pooled together for one sequencing run.

Sequencing on the MiSeq: In preparation for sequencing, combined libraries (up to 96 samples) are denatured with NaOH (0.2 N), diluted with hybridization buffer (10 mM TRIS, pH 8.5), and heat denatured (96° C.) for two minutes prior to loading on the MiSeq instrument. Since libraries are between 400-500 bps in length, the inexpensive MiSeq V2 Reagent Kit (500 cycles; REF 15033625; $360) can be utilized. The run time for this kit is approximately 24 hours.

### Example 2

Next-Generation Sequencing performed for Characterizing High-Prevalence Multi-Drug Resistance Mycobacterium tuberculosis Mutations. Next-generation sequencing (NGS) is the established method for genetic characterization of Mycobacterium tuberculosis (MTB) mutations conferring multi-drug resistance (MDR). However, NGS remains cost prohibitive and requires extensive library preparation. Using a panel of South African clinical isolates, a simplified, targeted PCR method for detecting high prevalence MDR-specific mutations in the rpoB (rifampin), katG (isoniazid), gyrA (Fluoroquinolone), and pncA (pyrazinamide) genes was utilized. Results were compared to those obtained by whole-genome sequencing (WGS).

Four targeted PCR assays were designed and optimized for: 1) rpoB including the rifampin-resistance determining region, 2) katG spanning the S-315-T resistance mutation, 3) gyrA including quinolone-resistance determining region, and 4) the complete pncA gene. Using Illumina MiSeq, targeted-PCR results were evaluated using clinical isolates (N=16) from South Africa and compared to WGS.

Of 16 clinical isolates analyzed by targeted sequencing, 12 (75%) harbored a S-450-L mutation, 3 (19%) contained a D-435-L, and one had a less prevalent Q-423-K rifampin resistance-conferring mutation in the rpoB. Analysis of KatG revealed 11 isolates (69%) harbored a S-315-T isoniazid-conferring mutation, 10 (63%) contained a Fluoroquinolone-resistance mutation in gyrA, and 11 (69%) contained a pncA gene mutation. All mutations obtained by targeted sequencing were confirmed by WGS.

Targeted NGS detected MDR-TB in 11 (69%) African isolates. In one MDR isolate, a less prevalent Q-423-K rpoB resistance-conferring mutation was detected. Targeted sequencing detects a broader range of resistance-conferring mutations than GeneXpert, and is more cost-effective, particularly in low-resource areas where culture or WGS are impractical.

Other embodiments and uses of the invention will be apparent to those skilled in the art from consideration of the specification and practice of the invention disclosed herein. All references cited herein, including all publications and U.S. and foreign patents and patent applications, are specifically and entirely incorporated by reference. The word comprising, where ever used, is intended to include the terms consisting and consisting essentially of. Furthermore, the word comprising, including, containing, and the like are not intended to be limiting. It is intended that the specification and examples be considered exemplary only with the true scope and spirit of the invention indicated by the following claims.

1. A method for determining microbial resistance comprising:
extracting microbial nucleic acid from a biological sample containing a microbe;
optionally, performing a quantitative PCR to increase quantity and/or assess the microbial nucleic acid;
providing a collection of primer pairs, wherein each primer pair contains a common sequence and a variable sequence, wherein the variable sequence hybridizes to multiple regions of the microbial nucleic acid responsible for the expression of resistance genes;
combining the microbial nucleic acid or nucleic acid after quantitative PCR with the collection of primers pairs in a PCR to generate a series of amplicons;
linking the series of amplicons to a unique sequence that is specific for the biological sample;
providing a second collection of primer pairs, wherein each primer pair contains a sequence that is complimentary to the unique sequence and a sequence that hybridizes to one or more of the amplicon;
combining the linked series of amplicons with the second collection of primers pairs in a PCR to generate a second series of amplicons;
sequencing the second series of amplicons;
comparing the sequences of the second series of amplicons with the sequence of a wild-type sequence of the microbial nucleic acid; and
identifying one or more mutations of the resistance genes of the biological sample.

2. The method of claim 1, wherein the microbial resistance comprises resistance to an antibiotic.

3. The method of claim 1, wherein the biological sample is a bodily fluid, a nasal discharge, a sputum sample, blood, a tissue sample, a biopsy, a culture sample, or a combination thereof.

4. The method of claim 1, wherein the microbe is a bacterium, a virus, a parasite, and/or a fungus.

5. The method of claim 1, wherein the microbe is Mycobacterium tuberculosis.

6. The method of claim 1, wherein the biological sample is collected in a transport medium containing guanidine, a reducing agent, a chelator, a nonionic detergent, and a buffer.

7. The method of claim 6, wherein the biological sample is sterilized.

8. The method of claim 1, wherein extracting is performed by chemical or mechanical treatment of the biological sample.

9. The method of claim 1, wherein the primer pairs of the collection and/or the second collection are each from about 20 to about 35 nucleotides in length.

10. The method of claim 1, wherein the common and/or the unique sequence is from about 8 to 15 nucleotides in length.

11. The method of claim 1, wherein each of the multiple regions of the microbial genome are each from about 2 kb to about 20 kb in length.

12. The method of claim 1, wherein the microbial genome contains four or more different antibiotic resistance genes.

13. The method of claim 12, wherein the antibiotic resistance genes include rpoB, katG, gyrA, and pncA of Mycobacterium tuberculosis.

14. The method of claim 1, wherein linking if performed by ligating the common sequence to the 5′ terminus of each amplicon of the series of amplicons.

15. The method of claim 1, wherein linking if performed by ligating the unique sequence to the 5′ terminus of each amplicon of the second series of amplicons.

16. The method of claim 1, wherein the polymerase chain reaction is an RT-PCR.

17. The method of claim 1, wherein the amplicons of the series of amplicons generated and/or second series of amplicons generated are from 50 to 1,000 nucleotides in length.

18. The method of claim 17, wherein the amplicons of the series of amplicons generated and/or second series of amplicons generated are from 100 to 500 nucleotides in length.

19. The method of claim 1, wherein the amplicons of the series of amplicons generated and/or second series of amplicons generated are diluted in a buffer and a portion of the diluted amplicons subjected to sequencing.

20. The method of claim 1, wherein sequencing is performed by ion torrent or next generation sequencing.

21. The method of claim 20, wherein sequencing of amplicons of the second series of amplicons is performed in one step.

22. The method of claim 1, which is performed in about 24 to about 36 hours.

23. The method of claim 1, which is performed in less than about 24 hours.

24. A method of treating a patient infected with Mycobacteria comprising:
performing the method of claim 1, wherein the biological sample is obtained from the patient; and
treating the patient with one or more drugs or drug combinations.

25. The method of claim 24, wherein the time period from performing the method to treating the patient is less than 48 hours.

26. The method of claim 25, wherein the time period from performing the method to treating the patient is less than 36 hours.

27. The method of claim 26, wherein the time period from performing the method to treating the patient is less than 24 hours.

28. A kit for the detection of Mycobacteria comprising:
a transport media for collection of a biological sample;
a collection of primer pairs for a PCR reaction, wherein each primer pair contain a common sequence and a variable sequence, wherein the variable sequence hybridizes to multiple regions of the microbial genome responsible for the expression of resistance genes;
a second collection of primer pairs, wherein each primer pair of the second collection contains a sequence that is complimentary to the unique sequence; and
a PCR mixture comprising: a heat-stable polymerase, deoxynucleotide tri phosphates comprising about equal amounts of dATP, dCTP, dGTP and/or dTTP; a chelating agent; a salt; a buffer; and a stabilizing agent.

29. A method for determining microbial resistance in multiple biological samples comprising:
extracting nucleic acid from each of the multiple biological samples, each containing the same microbe;
optionally, separately performing a quantitative PCR to increase quantity and/or assess the microbial nucleic acid of one or more of the multiple biological samples;
providing a collection of primer pairs, wherein each primer pair contain a common sequence and a variable sequence, wherein the variable sequence hybridizes to multiple regions of the microbial genome responsible for the expression of resistance genes;
separately combining each microbial nucleic acid extracted with the collection of primers pairs in a PCR to generate a series of amplicons for each biological sample;
separately linking each series of amplicons to a unique sequence that is specific for the biological sample;
providing second collections of primer pairs, one second collection for each series of amplicons generated, wherein each second collection contains a sequence that is complimentary to the unique sequence and a sequence that hybridizes to one or more of the amplicons;
separately combining the linked series of amplicons with the second collections in a PCR to generate a second series of amplicons for each biological sample;
pooling all the second series of amplicons for each biological sample generated;
sequencing the pooled amplicons;
comparing the sequences of the pooled amplicons with the sequence of a wild-type sequence of the microbial nucleic acid; and
identifying one or more mutations in the resistance genes for each of the biological samples.
